(19) United States
(12) Patent Application Publication
(10) Pub. No.: US 2013/0087579 A1
(43) Pub. Date: Apr. 11, 2013

(54) Sanitation dispenser

(71) Applicant: Shanina C. Knighton, Euclid, OH (US)

(72) Inventor: Shanina C. Knighton, Euclid, OH (US)

(21) Appl. No.: 13/068,906

(22) Filed: Oct. 11, 2011

(51) Int. Cl. B67D 7/06

(52) U.S. Cl. 222/180

(57) ABSTRACT

A sanitation dispenser includes: an adjustable attaching mechanism to secure a sanitation dispenser to an existing structural member within an environment of a person so that the sanitation dispenser is accessible to the person for use while maintaining a seated, inclined, or laid out position, a compartment with an opening for receiving a consumable sanitation product such that the consumable sanitation product can be dispensed from the compartment via a dispensing outlet and replenished via the opening, a connecting member extending from the adjustable attaching mechanism to the compartment to form the sanitation dispenser, and a cover mating with the opening in the compartment to enclose the consumable sanitation product within the compartment for use of the sanitation dispenser by the person.

### BACKGROUND

This invention relates to hand hygiene practices in the hospital and how we can bring more accountability upon the patient with a device that is meant specifically for them. Year after year, hospital acquired infections are growing with no end in sight because of the lack of containment and prevention in the hospital setting. When we look at the main reason for the spread of these germs which is lack of hand hygiene, we realize that this is stemming from bad habits that have been developed over ones career/life and in order to reverse these trends we will need to take measures that will go above and beyond traditional practices that are implemented now.

Immediate action needs to be done in the hand hygiene methods and behavioral changes in the healthcare system. Medical facilities such as hospitals, nursing homes, clinics, etc. have been hit hard with the rising infection costs because of a number of multi-resistant highly infectious and virulent super bugs such as Methicillin Resistant Staphylococcus Aureus (MRSA). MRSA is the fastest growing and the most deadly, it has risen from a 2% presence in staph infection studies in 1974 to 57% in 2003. Overall, hospital acquired infections affect about 2 million patients per year and have been estimated to cause around ninety thousand patient deaths annually; this costs the US $37-$45 billion per year. Knowing how simple yet important hand hygiene is on cutting down the spread of infection, compliance rates our below fifty percent for healthcare staff and there is very little to no research on the percentage for patients. Seventy percent of hospital acquired infections can be prevented and hand washing is the main contributor.

Patients who acquire these infections are extending their stay in the hospital on average 5 days. For example, a patient who comes in for a relatively small procedure that normally would not last more than a few days contracts MRSA, their stay can be extended a long period of time which will drive up healthcare cost. This also hurts the hospital because the patient's bed is occupied and will not be able to be used for another patient, which strains the hospital who is already under pressure to reduce waiting lists. This causes an issue for the family, because not only are they not profiting from the extended stay but also paying for treatment. This was a result of legislature passed called the Enactment law, signed Feb. 8, 2006.

Frequent washing of the hands with plain soap and water or with antimicrobial soap and water has been long recommended. Through guidelines and numerous research projects, it is now recommended that the healthcare workers use an alcohol-based hand rub to routinely clean their hands between patient contacts. These contacts include: touching the patient's skin or after touching equipment/furniture near the patient and after removing their gloves. Patients harbor these germs and they are usually given nothing to sanitize their hands. Patients do not have outlets when they are in the hospital because there is nothing for them to do. So they will blow their nose, sneeze, cough, use their urinal, eat, and change their surgical dressings all in the bed, which unless they are able to get to the sink to wash their hands, they will sit in those germs. There are numerous conspicuous hand sanitation areas for healthcare staff and families in the hospital: room sink, corridor dispenser, room dispenser, etc., but none addresses the patient besides the room sink. Through research by the Journal of the American Osteopathic Association, it was concluded that volumes of use for alcohol-based hand hygiene agents were significantly increased in daily consumption after conspicuous positioning of dispensers.

Another problem with the spread of hospital acquired infections is that health services are exposed to a great deal of liability and initial indicators are that the cost of litigation and compensation for health services settling the cases of patients that have contracted hospital acquired infections in their facilities are costing billions of dollars.

Therefore, this invention is aimed at addressing the shortcomings described above along with others that are apparent in this disclosure.

### SUMMARY

In one aspect, a sanitation dispenser is provided. In one embodiment, the sanitation dispenser includes: an adjustable attaching mechanism to secure a sanitation dispenser to an existing structural member within an environment of a person so that the sanitation dispenser is accessible to the person for use while maintaining a seated, inclined, or laid out position, a compartment with an opening for receiving a consumable sanitation product such that the consumable sanitation product can be dispensed from the compartment via a dispensing outlet and replenished via the opening, a connecting member extending from the adjustable attaching mechanism to the compartment to form the sanitation dispenser, and a cover mating with the opening in the compartment to enclose the consumable sanitation product within the compartment for use of the sanitation dispenser by the person.

In another aspect, a sanitation device is provided. In one embodiment, the sanitation device includes: a resiliently biased attaching mechanism to secure a sanitation device to an existing structural member of a hospital bed so that the sanitation device is accessible to a patient assigned to the hospital bed is maintaining a seated, inclined, or laid out position in the hospital bed, a compartment with an opening for receiving a consumable sanitation product such that the consumable sanitation product can be dispensed from the compartment via a dispensing outlet and replenished via the opening, a connecting member extending from the resiliently biased attaching mechanism to the compartment to form the sanitation device, and a cover mating with the opening in the compartment to enclose the consumable sanitation product within the compartment for use of the sanitation device by the patient.

In another aspect, sanitizing dispenser is provided. In one embodiment, the sanitizing dispenser includes: an adjustable attaching mechanism to secure a sanitizing dispenser to an existing structural member within an environment of a person so that the sanitizing dispenser is accessible to the person for use while maintaining a seated, inclined, or laid out position, a compartment with an opening for receiving a consumable sanitation product such that the consumable sanitation product can be dispensed from the compartment via a dispensing outlet and replenished via the opening, a connecting member extending from the adjustable attaching mechanism to the compartment to form the sanitizing dispenser, a cover mating with the opening in the compartment to enclose the consumable sanitation product within the compartment for use of the sanitizing dispenser by the person, and a dispensing mechanism in operative communication with the compartment to control dispensing of the consumable sanitation product via the dispensing outlet. In this embodiment, the dispensing mechanism includes at least one of a spout mechanism suitable for dispensing liquid, an opening suitable for dispensing wipes or tissues, a bulb mechanism suitable for dispensing liquid, a push button mechanism suitable for manual activation to dispense liquid, a lever mechanism suitable for manual activation to dispense liquid, and an optical sensor suitable for hands-free activation to dispense liquid.

### DESCRIPTION OF DRAWINGS

FIG. 1 provides a perspective view of an exemplary embodiment of a sanitation dispenser;

FIG. 2 provides a perspective view of another exemplary embodiment of a sanitation dispenser;

FIG. 3 provides a perspective view of yet another exemplary embodiment of a sanitation dispenser;

FIG. 4 provides a perspective view of still another exemplary embodiment of a sanitation dispenser;

FIG. 5 provides a perspective view of still yet another exemplary embodiment of a sanitation dispenser; and

FIG. 6 shows an example of an installation of an exemplary embodiment of a sanitation dispenser in a hospital room.

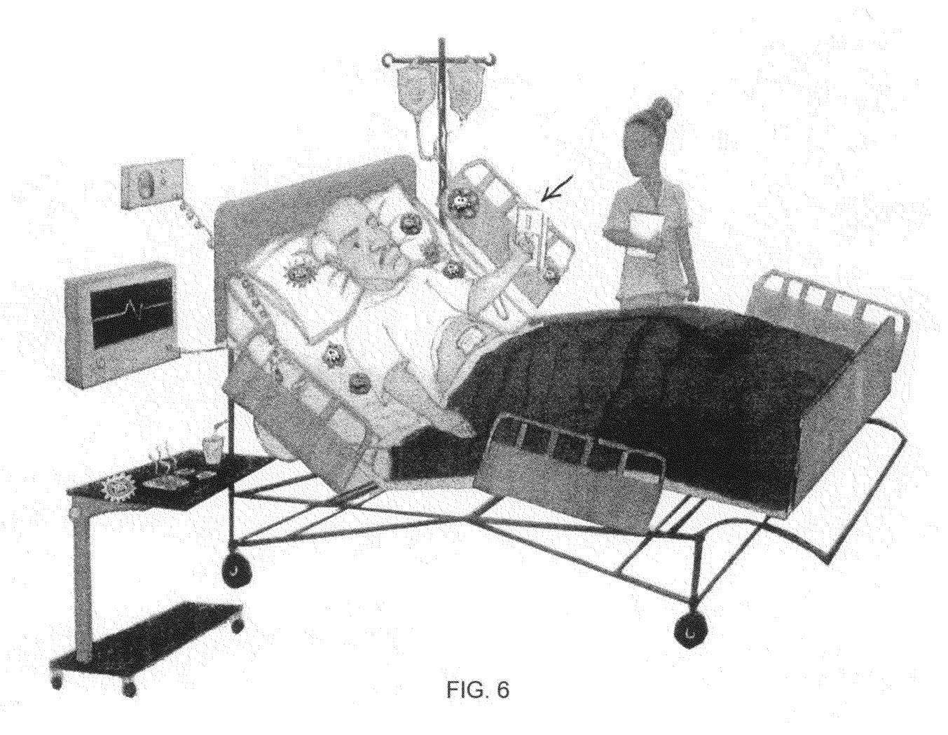

### DETAILED DESCRIPTION

In the hospital setting, there has been tremendous amount of research showing how the healthcare staff and family transmit germs and infections throughout the hospital because of negligence of hand hygiene. Very little research has been conducted about the patient and their vital role in the spread of these germs and hospital acquired infections. With this knowledge, I have created a hand sanitizer dispenser that is compact and meant for individual use. In one embodiment, a hand sanitation dispenser with a universal clamp/clip is attached to the rear of the dispenser so it will have the capability to affix itself to any bed rail, wheel chair, pole, etc.

Hand hygiene has been proven to be the easiest and most effective way to cut down on the spread of germs. Overall, with widely accepted usage of hand sanitizer dispensers, it has made it easier to attack the problem of hand washing/sanitation. There has also been research done on the location of the hand sanitation stations and if they are in conspicuous places then they will get used more often than in obscure locations. Hand sanitizer dispensers were created in order to bring the convenience of a sink to more conspicuous places. This invention has embodied that thought and developed it more to an individual purpose. The design of the dispenser was made compact because it is intended for hospital patient use in the bed, which is occupied by the user as well as bedside medical or miscellaneous equipment. The compact dispenser can be located in very close proximity to the user at all times so they will be constantly reminded about hand hygiene. In the development of this invention, the understanding that a change of cultural behavior needs to be set in place was the driving factor in creating this bedside sanitation dispenser.

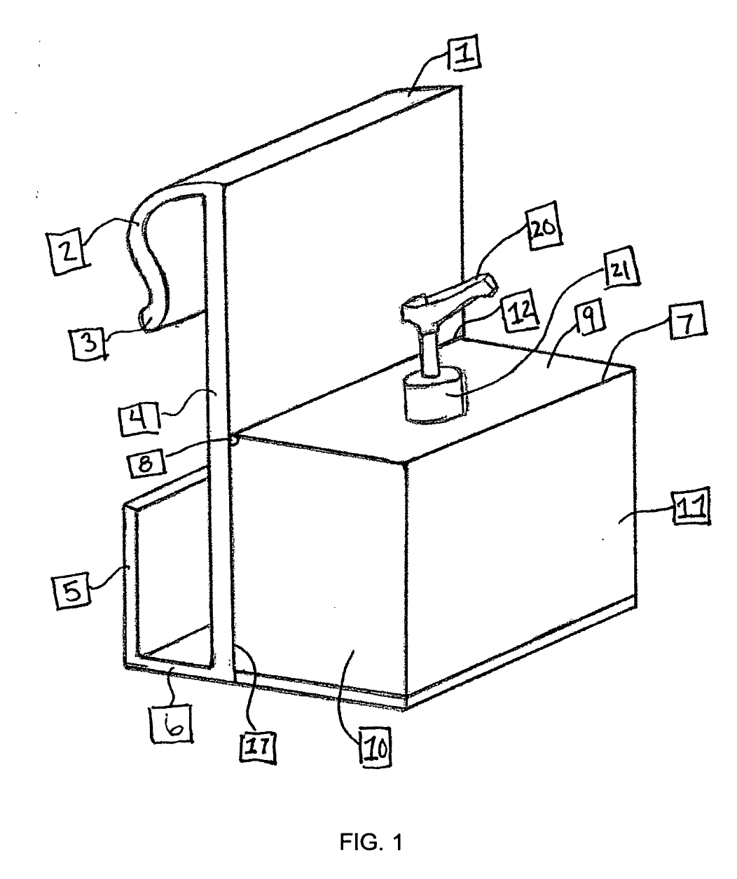

This apparatus will have capabilities to affix itself to any bed rail, arm chair, or any other location suitable to hold the product and will hold a sanitation bag to dispense hand sanitation solution or any other similar solution. FIG. 1 is a ¾ side view of one embodiment of this invention showing the hand sanitizing bag enclosed in the frontal compartment of the device. As shown, a refillable sanitation dispenser is comprised of a hand pump and nozzle on the top of the apparatus.

This is a flat portion of the clip (1) that extends out from the (4) back plate of the apparatus that supports the entire apparatus. (1) The flat portion allows the rail or arm of a chair to rest flatly against it, as the (2) groove design curves out to fit either the rail or the arm and the space between it and the back plate (4). The clip curves back in (3) to snuggly grasp either the rail or the arm. There is a tissue holder connected to the back plate (4) by the base (6) and the back wall (5). The front compartment is connected to the back plate (4) and encloses the sanitizer by 5 walls (6, 9, 10, 11, last side not shown). Numbers (7, 12, 17) are all adjacent joints where the walls meet. The lid (9) is connected to the back plate (4) by a hinge (8). In this figure, the sanitizer holder is equipped to handle a bottle which has the spout (20) and base (21) sticking out of the lid.

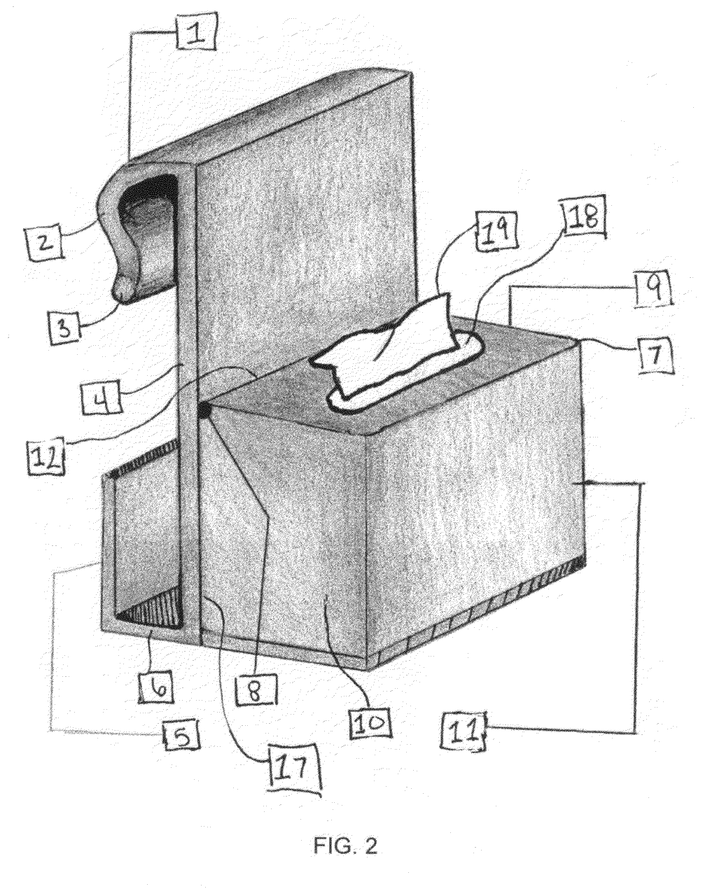

With the same attaching mechanism as FIG. 1, FIG. 2 is a ¾ side view of one embodiment of this invention showing the device containing non-alcoholic based, anti-microbial sanitation wipes that will be folded inside the storage compartment and dispense in an orderly fashion. The wipes are enclosed in the frontal compartment of the device that is able to clip to any rail (bed) or arm (chair).

This is a flat portion of the clip (1) that extends out from the (4) back plate of the apparatus that supports the entire apparatus. (1) The flat portion allows the rail or arm of a chair to rest flatly against it, as the (2) groove design curves out to fit either the rail or the arm and the space between it and the back plate (4). The clip curves back in (3) to snuggly grasp either the rail or the arm. There is a tissue holder connected to the back plate (4) by the base (6) and the back wall (5). The front compartment is connected to the back plate (4) and encloses the sanitizer by 5 walls (6, 9, 10, 11, last side not shown). Numbers (7, 12, 17) are all adjacent joints where the walls meet. The lid (9) is connected to the back plate (4) by a hinge (8). In this figure, there is an opening (18) in the lid (9) and the wipes (19) are discharged from there.

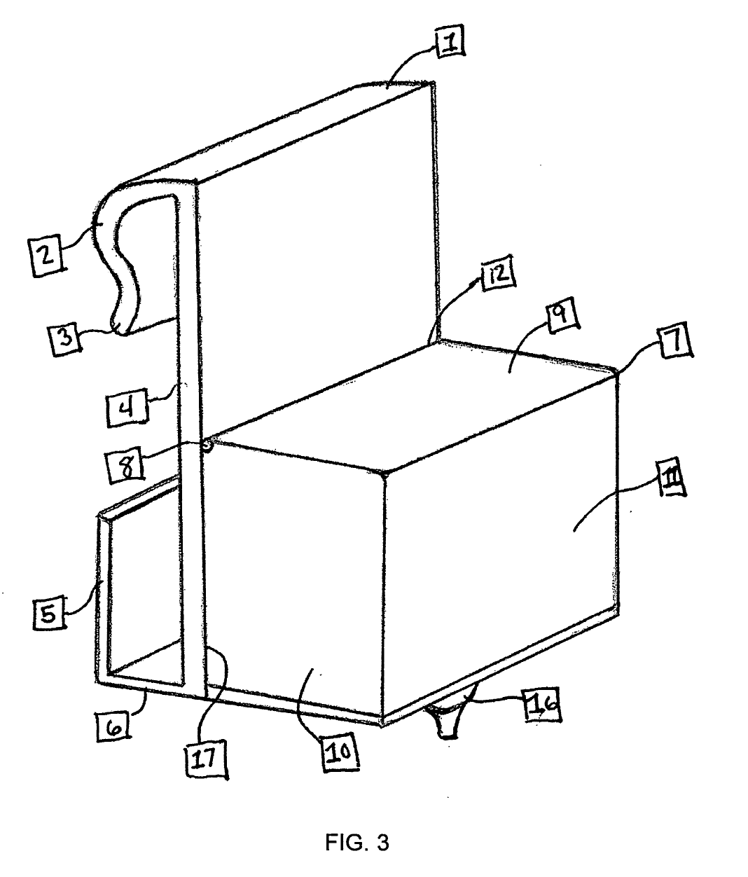

With the same attaching mechanism as FIGS. 1 and 2, FIG. 3 is a ¾ side view of one embodiment of this invention showing the bulb dispensing mechanism for the liquid coming from underneath front compartment of the device. The device will be activated when the user pushes on the bulb to dispense liquid through the nozzle.

This is a flat portion of the clip (1) that extends out from the (4) back plate of the apparatus that supports the entire apparatus. (1) The flat portion allows the rail or arm of a chair to rest flatly against it, as the (2) groove design curves out to fit either the rail or the arm and the space between it and the back plate (4). The clip curves back in (3) to snuggly grasp either the rail or the arm. There is a tissue holder connected to the back plate (4) by the base (6) and the back wall (5). The front compartment is connected to the back plate (4) and encloses the sanitizer by 5 walls (6, 9, 10, 11, last side not shown). Numbers (7, 12, 17) are all adjacent joints where the walls meet. The lid (9) is connected to the back plate (4) by a hinge (8). In this figure, that hand sanitizer bulb (16) separates from the bottom plate (6) to dispense the hand sanitizer.

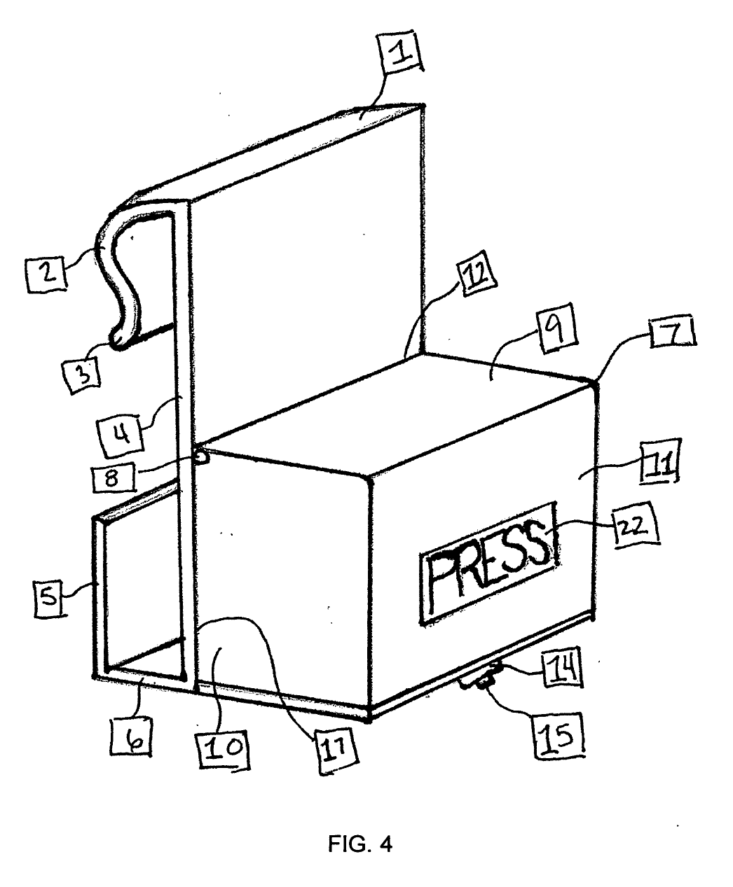

With the same attaching mechanism as FIGS. 1-3, FIG. 4 is a ¾ side view of one embodiment of this invention showing the press feature on the frontal wall of housing compartment and the spout to dispense the hand sanitizer will protrude from underneath the housing compartment of the device. As shown, the device will have the function of dispensing solution by pressing an activation button on the front.

This is a flat portion of the clip (1) that extends out from the (4) back plate of the apparatus that supports the entire apparatus. (1) The flat portion allows the rail or arm of a chair to rest flatly against it, as the (2) groove design curves out to fit either the rail or the arm and the space between it and the back plate (4). The clip curves back in (3) to snuggly grasp either the rail or the arm. There is a tissue holder connected to the back plate (4) by the base (6) and the back wall (5). The front compartment is connected to the back plate (4) and encloses the sanitizer by 5 walls (6, 9, 10, 11, last side not shown). Numbers (7, 12, 17) are all adjacent joints where the walls meet. The lid (9) is connected to the back plate (4) by a hinge (8). In this figure, there is a press button (22) on the front wall (11) that when activated, will cause the hand sanitizer to dispense from the spout or pump (14 and 15).

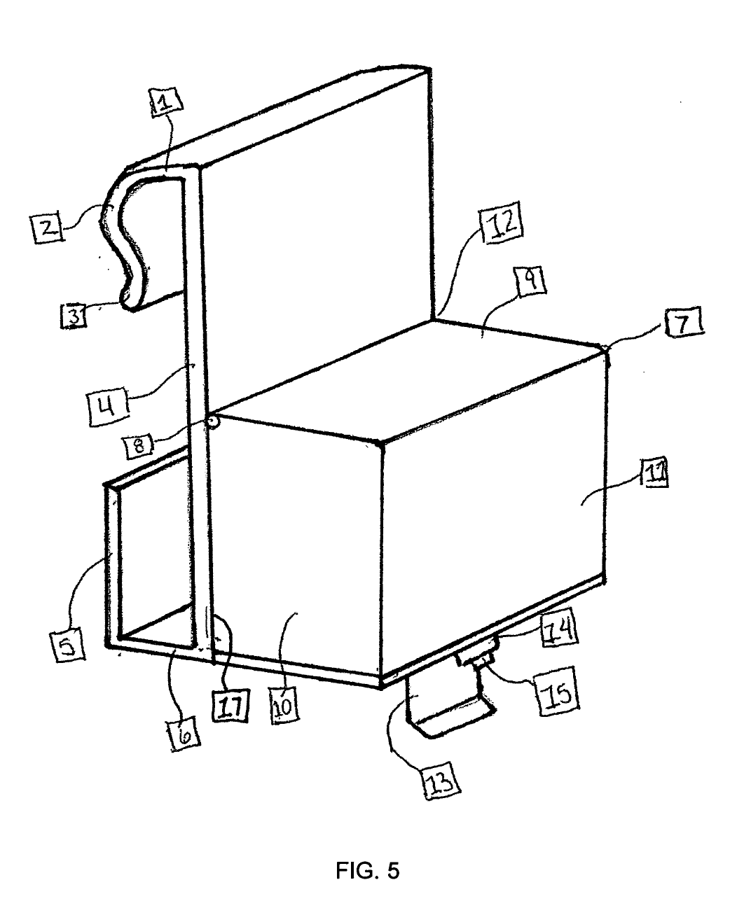

With the same attaching mechanism as FIGS. 1-4, FIG. 5 is a ¾ side view of one embodiment of this invention showing a dispensing mechanism wherein the operator pulls a lever underneath the compartment, dispensing a liquid from the spout in front of the activating lever.

This is a flat portion of the clip (1) that extends out from the (4) back plate of the apparatus that supports the entire apparatus. (1) The flat portion allows the rail or arm of a chair to rest flatly against it, as the (2) groove design curves out to fit either the rail or the arm and the space between it and the back plate (4). The clip curves back in (3) to snuggly grasp either the rail or the arm. There is a tissue holder connected to the back plate (4) by the base (6) and the back wall (5). The front compartment is connected to the back plate (4) and encloses the sanitizer by 5 walls (6, 9, 10, 11, last side not shown). Numbers (7, 12, 17) are all adjacent joints where the walls meet. The lid (9) is connected to the back plate (4) by a hinge (8). In this figure, there is a pull activation lever (13) that is located underneath the base plate (6) of the sanitizer compartment. There is also the same spout or pump (14 and 15) from the press button sanitizer dispenser.

In summary, the sanitation dispenser will be user safety oriented and shall have curved or rounded edges. It shall be compact for the use of individuals in small enclosures and the device will have antimicrobial solutions mixed in with the plastic to reduce cross or recontamination of the user. The frontal holding compartment will have the function to enclose a liquid solution and lock the top lid in place in order to cut down on an individual tampering with the components inside. The sanitation device will be able to firmly lock the solution bag in the compartment, so it will be able to be used for ideal dispensing. The sanitation device will have a universal holding compartment on the back of the apparatus that will have the capabilities to hold tissue, trash bag, or any other small utility item that the user deems necessary for their use. The sanitation devices frontal compartment will have the capabilities to rotate 90 degrees to the left and to the right; this is done to account for bed tilt or any angle of an attachable rail.

In one exemplary embodiment, a sanitation dispenser includes an adjustable attaching mechanism, a compartment, a connecting member, and a cover. The adjustable attaching mechanism secures the sanitation dispenser to an existing structural member within an environment of a person so that the sanitation dispenser is accessible to the person for use while maintaining a seated, inclined, or laid out position. The compartment includes an opening for receiving a consumable sanitation product such that the consumable sanitation product can be dispensed from the compartment via a dispensing outlet and replenished via the opening. The connecting member extends from the adjustable attaching mechanism to the compartment to form the sanitation dispenser. The cover mates with the opening in the compartment to enclose the consumable sanitation product within the compartment for use of the sanitation dispenser by the person.

In another embodiment of the sanitation dispenser, the adjustable attaching mechanism includes a resiliently biased clamping mechanism to secure the sanitation dispenser to the existing structural member. In an alternate embodiment, the adjustable attaching mechanism includes a resiliently biased clip mechanism to secure the sanitation dispenser to the existing structural member.

In yet another embodiment of the sanitation dispenser, the existing structural member includes a bed, a chair, a table, a pole, a post, a handrail, a grab handle, a walker, or another suitable existing structural member within the environment of the person. In still another embodiment, the sanitation dispenser is constructed of materials that include an antimicrobial agent to reduce contamination from prior use. In still yet another embodiment, the sanitation dispenser also includes a locking mechanism suitable for securing the cover to the compartment in a manner suitable for preventing the person from tampering with the consumable sanitation product.

In another embodiment, the sanitation dispenser also includes a utility fixture extending from the connecting member to facilitate attachment or storage of an accessory associated with the sanitation dispenser. In a further embodiment, the accessory includes a tissue, a towel, a trash receptacle, or another suitable accessory in any suitable combination.

In yet another embodiment of the sanitation dispenser, the compartment is attached to the connecting member in a manner that permits rotation of the compartment in relation to the adjustable attaching mechanism to facilitate attachment of the sanitation dispenser to existing structural members having different orientations while maintaining a desired orientation of the dispensing outlet for use of the sanitation dispenser. In still another embodiment of the sanitation dispenser, the compartment is attached to the connecting member in a manner that permits rotation of the compartment in relation to rotational movement of the existing structural member to which the sanitation dispenser is secured by the adjustable attaching mechanism from an initial orientation to a different orientation in order to maintain a desired orientation of the dispensing outlet for use of the sanitation dispenser.

In still yet another embodiment, the sanitation dispenser also includes a dispensing mechanism in operative communication with the compartment to control dispensing of the consumable sanitation product via the dispensing outlet. In a further embodiment, the dispensing mechanism includes a spout mechanism suitable for dispensing liquid, an opening suitable for dispensing wipes or tissues, a bulb mechanism suitable for dispensing liquid, a push button mechanism suitable for manual activation to dispense liquid, a lever mechanism suitable for manual activation to dispense liquid, an optical sensor suitable for hands-free activation to dispense liquid, or another suitable dispensing mechanism.

In another embodiment of the sanitation dispenser, the consumable sanitation product includes a sanitizing foam, a sanitizing gel, a sanitizing lotion, a sanitizing wipe, a sanitizing solution, a sanitizing liquid, a water-based liquid, or another suitable consumable sanitation product.

In one exemplary embodiment, a sanitation device includes a resiliently biased attaching mechanism, a compartment, a connecting member, and a cover. The resiliently biased attaching mechanism secures a sanitation device to an existing structural member of a hospital bed so that the sanitation device is accessible to a patient assigned to the hospital bed while the patient is maintaining a seated, inclined, or laid out position in the hospital bed. The compartment includes an opening for receiving a consumable sanitation product such that the consumable sanitation product can be dispensed from the compartment via a dispensing outlet and replenished via the opening. The connecting member extends from the resiliently biased attaching mechanism to the compartment to form the sanitation device. The cover mates with the opening in the compartment to enclose the consumable sanitation product within the compartment for use of the sanitation device by the patient.

In another embodiment of the sanitation device, the existing structural member includes a bed rail, a table, a pole, a post, a handrail, a grab handle, or another suitable existing structural member of the hospital bed. In yet another embodiment of the sanitation device, the compartment is attached to the connecting member in a manner that permits rotation of the compartment in relation to the resiliently biased attaching mechanism to facilitate attachment of the sanitation device to existing structural members having different orientations while maintaining a desired orientation of the dispensing outlet for use of the sanitation device. In still another embodiment of the sanitation device, the compartment is attached to the connecting member in a manner that permits rotation of the compartment in relation to rotational movement of the existing structural member to which the sanitation device is secured by the resiliently biased attaching mechanism from an initial orientation to a different orientation in order to maintain a desired orientation of the dispensing outlet for use of the sanitation device.

In one exemplary embodiment, a sanitizing dispenser includes an adjustable attaching mechanism, a compartment, a connecting member, a cover mating, and a dispensing mechanism. The adjustable attaching mechanism secures a sanitizing dispenser to an existing structural member within an environment of a person so that the sanitizing dispenser is accessible to the person for use while maintaining a seated, inclined, or laid out position. The compartment includes an opening for receiving a consumable sanitation product such that the consumable sanitation product can be dispensed from the compartment via a dispensing outlet and replenished via the opening. The connecting member extends from the adjustable attaching mechanism to the compartment to form the sanitizing dispenser. The cover mates with the opening in the compartment to enclose the consumable sanitation product within the compartment for use of the sanitizing dispenser by the person. The dispensing mechanism is in operative communication with the compartment to control dispensing of the consumable sanitation product via the dispensing outlet. The dispensing mechanism includes a spout mechanism suitable for dispensing liquid, an opening suitable for dispensing wipes or tissues, a bulb mechanism suitable for dispensing liquid, a push button mechanism suitable for manual activation to dispense liquid, a lever mechanism suitable for manual activation to dispense liquid, an optical sensor suitable for hands-free activation to dispense liquid, or another suitable dispensing mechanism.

In another embodiment of the sanitizing dispenser, the compartment is attached to the connecting member in a manner that permits rotation of the compartment in relation to the adjustable attaching mechanism to facilitate attachment of the sanitizing dispenser to existing structural members having different orientations while maintaining a desired orientation of the dispensing outlet for use of the sanitizing dispenser. In yet another embodiment of the sanitizing dispenser, the compartment is attached to the connecting member in a manner that permits rotation of the compartment in relation to rotational movement of the existing structural member to which the sanitizing dispenser is secured by the adjustable attaching mechanism from an initial orientation to a different orientation in order to maintain a desired orientation of the dispensing outlet for use of the sanitizing dispenser.

The above description merely provides a disclosure of particular embodiments of the invention and is not intended for the purposes of limiting the same thereto. As such, the invention is not limited to only the above-described embodiments. Rather, it is recognized that one skilled in the art could conceive alternative embodiments that fall within the scope of the invention.

We claim:

1. A sanitation dispenser, comprising:
an adjustable attaching mechanism to secure a sanitation dispenser to an existing structural member within an environment of a person so that the sanitation dispenser is accessible to the person for use while maintaining a seated, inclined, or laid out position;
a compartment with an opening for receiving a consumable sanitation product such that the consumable sanitation product can be dispensed from the compartment via a dispensing outlet and replenished via the opening;
a connecting member extending from the adjustable attaching mechanism to the compartment to form the sanitation dispenser; and
a cover mating with the opening in the compartment to enclose the consumable sanitation product within the compartment for use of the sanitation dispenser by the person.

2. The sanitation dispenser of claim 1, the adjustable attaching mechanism comprising:
a resiliently biased clamping mechanism to secure the sanitation dispenser to the existing structural member.

3. The sanitation dispenser of claim 1, the adjustable attaching mechanism comprising:
a resiliently biased clip mechanism to secure the sanitation dispenser to the existing structural member.

4. The sanitation dispenser of claim 1 wherein the existing structural member includes at least one of a bed, a chair, a table, a pole, a post, a handrail, a grab handle, and a walker.

5. The sanitation dispenser of claim 1 wherein the sanitation dispenser is constructed of materials that include an antimicrobial agent to reduce contamination from prior use.

6. The sanitation dispenser of claim 1, further comprising:
a locking mechanism suitable for securing the cover to the compartment in a manner suitable for preventing the person from tampering with the consumable sanitation product.

7. The sanitation dispenser of claim 1, further comprising:
a utility fixture extending from the connecting member to facilitate attachment or storage of an accessory associated with the sanitation dispenser.

8. The sanitation dispenser of claim 7 wherein the accessory includes at least one of a tissue, a towel, and a trash receptacle.

9. The sanitation dispenser of claim 1 wherein the compartment is attached to the connecting member in a manner that permits rotation of the compartment in relation to the adjustable attaching mechanism to facilitate attachment of the sanitation dispenser to existing structural members having different orientations while maintaining a desired orientation of the dispensing outlet for use of the sanitation dispenser.

10. The sanitation dispenser of claim 1 wherein the compartment is attached to the connecting member in a manner that permits rotation of the compartment in relation to rotational movement of the existing structural member to which the sanitation dispenser is secured by the adjustable attaching mechanism from an initial orientation to a different orientation in order to maintain a desired orientation of the dispensing outlet for use of the sanitation dispenser.

11. The sanitation dispenser of claim 1, further comprising:
a dispensing mechanism in operative communication with the compartment to control dispensing of the consumable sanitation product via the dispensing outlet.

12. The sanitation dispenser of claim 11 wherein the dispensing mechanism includes at least one of a spout mechanism suitable for dispensing liquid, an opening suitable for dispensing wipes or tissues, a bulb mechanism suitable for dispensing liquid, a push button mechanism suitable for manual activation to dispense liquid, a lever mechanism suitable for manual activation to dispense liquid, and an optical sensor suitable for hands-free activation to dispense liquid.

13. The sanitation dispenser of claim 1 wherein the consumable sanitation product includes at least one of a sanitizing foam, a sanitizing gel, a sanitizing lotion, a sanitizing wipe, a sanitizing solution, a sanitizing liquid, and a water-based liquid.

14. A sanitation device, comprising:
a resiliently biased attaching mechanism to secure a sanitation device to an existing structural member of a hospital bed so that the sanitation device is accessible to a patient assigned to the hospital bed while the patient is maintaining a seated, inclined, or laid out position in the hospital bed;
a compartment with an opening for receiving a consumable sanitation product such that the consumable sanitation product can be dispensed from the compartment via a dispensing outlet and replenished via the opening;
a connecting member extending from the resiliently biased attaching mechanism to the compartment to form the sanitation device; and
a cover mating with the opening in the compartment to enclose the consumable sanitation product within the compartment for use of the sanitation device by the patient.

15. The sanitation device of claim 14 wherein the existing structural member includes at least one of a bed rail, a table, a pole, a post, a handrail, and a grab handle.

16. The sanitation device of claim 14 wherein the compartment is attached to the connecting member in a manner that permits rotation of the compartment in relation to the resiliently biased attaching mechanism to facilitate attachment of the sanitation device to existing structural members having different orientations while maintaining a desired orientation of the dispensing outlet for use of the sanitation device.

17. The sanitation device of claim 14 wherein the compartment is attached to the connecting member in a manner that permits rotation of the compartment in relation to rotational movement of the existing structural member to which the sanitation device is secured by the resiliently biased attaching mechanism from an initial orientation to a different orientation in order to maintain a desired orientation of the dispensing outlet for use of the sanitation device.

18. A sanitizing dispenser, comprising:
an adjustable attaching mechanism to secure a sanitizing dispenser to an existing structural member within an environment of a person so that the sanitizing dispenser is accessible to the person for use while maintaining a seated, inclined, or laid out position;
a compartment with an opening for receiving a consumable sanitation product such that the consumable sanitation product can be dispensed from the compartment via a dispensing outlet and replenished via the opening;
a connecting member extending from the adjustable attaching mechanism to the compartment to form the sanitizing dispenser;
a cover mating with the opening in the compartment to enclose the consumable sanitation product within the compartment for use of the sanitizing dispenser by the person; and
a dispensing mechanism in operative communication with the compartment to control dispensing of the consumable sanitation product via the dispensing outlet;
wherein the dispensing mechanism includes at least one of a spout mechanism suitable for dispensing liquid, an opening suitable for dispensing wipes or tissues, a bulb mechanism suitable for dispensing liquid, a push button mechanism suitable for manual activation to dispense liquid, a lever mechanism suitable for manual activation to dispense liquid, and an optical sensor suitable for hands-free activation to dispense liquid.

19. The sanitizing dispenser of claim 18 wherein the compartment is attached to the connecting member in a manner that permits rotation of the compartment in relation to the adjustable attaching mechanism to facilitate attachment of the sanitizing dispenser to existing structural members having different orientations while maintaining a desired orientation of the dispensing outlet for use of the sanitizing dispenser.

20. The sanitizing dispenser of claim 18 wherein the compartment is attached to the connecting member in a manner that permits rotation of the compartment in relation to rotational movement of the existing structural member to which the sanitizing dispenser is secured by the adjustable attaching mechanism from an initial orientation to a different orientation in order to maintain a desired orientation of the dispensing outlet for use of the sanitizing dispenser.
